(19) United States
(12) Patent Application Publication
(10) Pub. No.: US 2008/0040460 A1
(43) Pub. Date: Feb. 14, 2008

(54) METHOD AND SYSTEM FOR COMMUNICATION

(75) Inventors: Vinayakumar Oruvilakott, Kozhakkottur (IN); Shourya sarcar, Bangalore (IN); Smita Singh, Bangalore (IN); Ferry Tamtoro, Oconomowoc, WI (US); Ashish Ranjan, Patna (IN)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/427,634

(22) Filed: Jun. 29, 2006

(51) Int. Cl. A61B 5/05; G06F 15/177

(52) U.S. Cl. 709/221; 600/410

(57) ABSTRACT

A method of communicating in a communication system and the communication system are provided. The communication system comprises at least one imaging station with multiple network interfaces. Each network interface of the imaging station is identified with a network address. The method comprises steps of selecting a first network interface available for communication in an imaging station and configuring the imaging station to communicate via the first network interface. The method further comprises a step of communicating at least one imaging data from the imaging station via the first network interface.

### FIELD OF INVENTION

The invention generally relates to a transfer of imaging data in a communication system and more particularly to a transfer of a Digital Imaging and Communication in Medicine (DICOM) compliant imaging data via a communication system.

### BACKGROUND OF THE INVENTION

A communication system comprises multiple stationary or mobile-networked devices. Examples of such mobile-networked devices are personal digital assistants, notebooks, laptop computers and medical imaging stations. Many mobile-networked devices comprise multiple network interfaces such as a wired network interface and a wireless network interface. In a networked environment it is possible that the multiple network interfaces are connected over a single link. For example a mobile imaging station can be connected to a hospital server via a wired network such as a local area network (LAN) or a wireless access point.

Telemedicine is generally the electronic transmission of a medical imaging data from one imaging station to another for purposes of interpretation and consultation. Two imaging stations can be placed within a hospital environment or remotely. Such imaging stations can be connected via a LAN, a WAN or a wireless communication interface. The imaging station can communicate the imaging data to another imaging station in the communication system via a DICOM communication. For the imaging station to perform the DICOM communication, a network address is required. In an imaging station with multiple network interfaces for DICOM connectivity, for example a mobile radio diagnosis (RAD) system with a wired network interface and a wireless network interface, a single network address is provided to multiple network interfaces. A standard implementation would require the user to manually disable a first network interface and enable a second network interface and reconfigure the imaging station to communicate via the second network interface as necessary.

Considering that the imaging station can be a mobile imaging station, an operator may need to move the imaging station from room to room as part of an exam sequence. During the move, for example the operator can be performing DICOM operations on the wireless network interface. The operator upon entering an area with no wireless coverage or when the operator decides to get the performance advantage of the wired network interface, the operator may opt for switching to the wired network interface. Thus it may be desirable to switch the network interface as the network interfaces are on the same link. Typically, such a switching is manually performed by closing down the communication on an existing network interface and setting up the communication on a desired network interface. The process of switching is cumbersome and time consuming as the existing communication path has to be closed and the desired communication path has to be setup. Furthermore, there exists a risk of losing the imaging data during closing down the existing communication path.

Therefore, it is desirable for the imaging station to provide seamless transition between multiple network interfaces such that no operator involvement is necessary. This may improve the fast-pace workflow needed in the hospital environments. Hence there exists a need to provide a method of switching the network interface seamlessly without the operator involvement.

### SUMMARY OF THE INVENTION

The above-mentioned shortcomings, disadvantages and problems are addressed herein which will be understood by reading and understanding the following specification.

In one embodiment, the invention provides a method of communicating in a communication system comprising an imaging station. The imaging station comprises multiple network interfaces. The network interface is one of a wired network interface and a wireless network interface. Each network interface is identified with a network address. The network address comprises at least one of an Internet Protocol (IP) address, an Application Entity Title (AETitle) and a port number. The method comprises a step of maintaining a configuration table at the imaging station. The configuration table comprises a mapping of each network interface with a corresponding network address. The method further comprises steps of selecting a first network interface available for communication in the imaging station, configuring the imaging station to communicate via the first network interface and communicating at least one imaging data from the imaging station via the first network interface. The method further comprises selecting a second network interface available for communication in the imaging station, and automatically reconfiguring the imaging station to communicate via the second network interface. Further, the method of communicating comprises retransmitting the imaging data upon receiving an interrupt, the interrupt indicating an unsuccessful transmission.

Another embodiment of the invention provides a communication system comprising an imaging station with multiple network interfaces. The imaging station is configured to maintain a configuration table mapping each of the network interfaces with a corresponding network address. The imaging station is further configured to select a first network interface available for communication, configure itself to communicate via the first network interface and communicate at least one imaging data via the first network interface. The imaging station is further configured to automatically switch from the first network interface to a second network interface available for communication. The imaging station is configured to automatically switch from communicating imaging data via the first network interface to communicating imaging data via the second network interface upon configuring itself to communicate via the second network interface. The imaging station can further be configured to retransmit the imaging data upon receiving an interrupt. At least one imaging station in the communication system is configured to communicate with another imaging station via a DICOM communication.

Yet another embodiment of the invention provides a computer program product stored in one or more computer readable media for communicating in a communication system comprising an imaging station with multiple network interfaces. The computer program product comprises a routine for selecting a first network interface available for communication in the imaging station, a routine for configuring the imaging station to communicate via the first network interface and a routine for communicating at least one imaging data from the imaging station via the first network interface.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a flow diagram of a method of communicating in a communication system in an embodiment of the invention;

FIG. 2 shows a flow diagram of a method of communicating in a communication system in another embodiment of the invention;

FIG. 3 shows a block diagram of a communication system in an embodiment of the invention.

### DETAILED DESRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

In various embodiments of the invention, a method of communicating in a communication system and a communication system are described. However, the embodiments are not limited and may be implemented in connection with different applications. The application of the invention can be extended to other areas, for example a DICOM communication system. The invention provides a broad concept of a seamless switching of network traffic between the network interfaces of a networked device, which can be adapted in a similar networked environment. The design can be carried further and implemented in various forms and specifications.

A communication system can comprise one or more imaging stations. The imaging stations in the communication system can be connected via a network for example a LAN, a WAN, PACS or a hospital network. Each imaging station may optionally acquire, transmit, display, manipulate, store and/or print an imaging data. Each imaging station can comprise one of an imaging server and an imaging modality. The imaging server can be a database management system for storing and managing the imaging data. The imaging modality can acquire, display, manipulate and store the imaging data. The imaging modality can include a magnetic resonance imaging (MRI) system, computed tomography (CT) system, ultrasonography (US) system, nuclear medicine (NM) system, digital fluorography system, computer radiography (CR) system, digitized radiography (DR) system, data image acquisition equipments used for radiofluoroscopy, angiography, such as x-ray angiography and heart scanning and secondary capture devices for video, endoscopy, microscopy, and photography, such as digital cameras, scanners, electrocardiogram (ECG) machines, and the like. One skilled in the art shall however appreciate that, the examples of the imaging modality are not limited to the examples mentioned above and the invention shall have full scope of the claims.

The imaging data captured by the imaging modality may take numerous forms, including text, images, video, audio dictation, and waveform data. Other medical imaging data may include 3-D volume data; series data for all clinical data in a medical series, e.g., coronal slices vs. axial slices in a CT exam or echoes as T1 slices vs. T2 slices in an MRI exam; annotation data for notes made by a practitioner, usually relating to the clinical data; and background data such as patient history and/or physical examination information.

The imaging data generated or stored in the imaging station can be transmitted to another imaging station in the communication system. In one scenario, a first imaging station transmitting the imaging data and a second imaging station receiving the imaging data can be placed physically close to each other such as within a local area network (LAN). In another scenario the first imaging station and the second imaging stations can be wirelessly connected through a transmitter and a receiver pair. The association between the first imaging station and the second imaging station can be a dedicated connection, such as a public network like a virtual private network or the Internet. The connection may be a telephone line, a serial line Internet protocol (SLIP), point-to-point protocol (PPP), an XDSL link, a satellite or other wireless link, a cable modem, ATM network connection, an ISDN line, a DSL line, or other communication link. One skilled in the art shall however appreciate that, the examples of the communication link between the first imaging station and the second imaging station is not limited to the examples mentioned above and the invention shall have full scope of the claims.

The imaging station can comprise the imaging modality and a transceiver to communicate the imaging data. The transceiver can be a device, such as an IP host, computer, server, a firewall or a router. The transceiver may be connected to the imaging modality through an Ethernet connection, serial interfaces, parallel interfaces, RS422 and/or RS432 interfaces, Livewire interfaces, IEEE-1394 serial busses, token ring and/or other local area networks, universal serial buses, PCI buses and wireless (e.g., infrared) connections, and the like.

The transceiver receives the imaging data from the imaging modality of the first imaging station and transmits the imaging data through the network connection to the second imaging station. It should be noted that the scope of the invention anticipates any number of imaging modalities, transceivers and imaging stations configured in accordance herewith and arranged in various fashions.

The imaging data may be formatted to be in compliance with several medical standards, for example DICOM and HL7 Standards. Thus, each imaging station can comprise a DICOM converter to convert the imaging data to a DICOM compliant imaging data. The DICOM converter may be a stand-alone device, or alternatively, may be an integral part of the imaging station to control the operations of the imaging modality.

The imaging modality can be coupled to the transceiver through the DICOM converter. The DICOM converter may receive the imaging data from imaging modality and convey the DICOM compliant imaging data to the transceiver. The transceiver may process the imaging data and send the imaging data to the second imaging station across the network. The imaging data leaving the transceiver may no longer be compliant with DICOM Standards, but may become DICOM compliant again by the transceiver of the second imaging station restoring appropriate DICOM information, to the imaging data prior to transferring the imaging data to the imaging modality of the second imaging station. The second imaging station may optionally transmit, display, manipulate, store and/or print the imaging data.

The transceiver of each imaging station may further include an acknowledgement unit configured to send and/or receive acknowledgements pertaining to a communicated imaging data. The acknowledgement unit considers an acknowledgment received within a predefined time interval as representing a successful transmission of the imaging data. Alternatively, a processing unit coupled to the acknowledgment unit can generate an interrupt, when the acknowledgement unit does not receive an acknowledgement for the imaging data within the predefined time interval. The imaging station upon receiving the interrupt considers the imaging data to be lost and retransmits the imaging data.

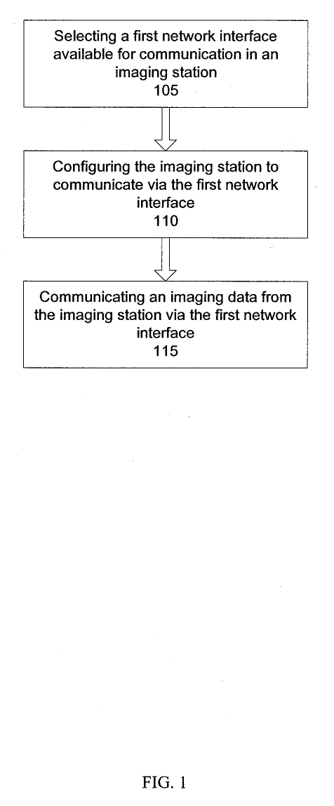

In an embodiment, the invention describes a method of communicating in a communication system as shown at FIG. 1. More specifically the invention describes a method to handle multiple network interfaces used in an imaging station of the communication system. The method comprises steps of selecting a first network interface available for communication step 105, configuring the imaging station to communicate via the first network interface step 110 and communicating one or more imaging data from the imaging station via the first network interface step 115.

The imaging data to be transmitted from the imaging station are put in the form of a queue. In order to communicate the imaging data, the imaging station needs to form an association with the second imaging station via the network interface. The imaging station detects one or more available network interfaces, the available network interface being a network interface available for communication and selects one of the available network interface based on predefined parameters. The predefined parameters can be for example signal strength, cost and reliability. One skilled in the art shall however appreciate that, the examples of the predefined parameters are not limited to the examples mentioned above and the invention shall have full scope of the claims.

Upon selecting the first network interface the imaging station configures itself to communicate via the first network interface. The imaging station in the process of configuring itself to communicate via the first network interface fetches the network address of the first network interface from a configuration table. The configuration table maintained at the imaging station provides a mapping of each network interface with a corresponding network address. A number of platforms provide a method of configuring multiple network interfaces with the corresponding network address. The imaging station upon configuring itself to the corresponding network address of the first network interface, communicates one or more imaging data to the second imaging station.

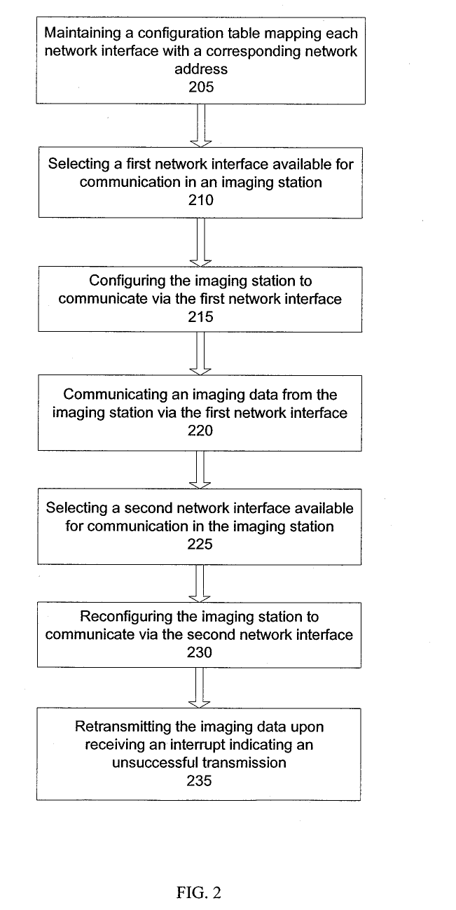

FIG. 2 shows a method of communicating in a communication system in another embodiment of the invention. The method comprises steps of maintaining a configuration table at the imaging station shown at step 205. The step of maintaining a configuration table is a one-time configuration. The configuration table can be created prior to starting the communication in the imaging station and can be updated based on a requirement. The requirement can be set on timely basis such as upon completing a predetermined time interval or upon receiving a signal indicating a change in the network address of the network interface.

The step of detecting an available network interface, selecting the first network interface, the first network interface being available for communication 210 and configuring the imaging station to communicate via the first network interface 215 can repeat as long as the imaging station is running. Upon configuring the imaging station to communicate via the first network interface, one or more imaging data can be communicated to another imaging station in the communication system step 220. The method of communicating further comprises selecting a second network interface available for communication in the imaging station step 225, and automatically reconfiguring the imaging station to communicate via the second network interface step 230. As a preliminary matter, the definition of the term “first” and “second” for the purposes of the description and the appended claims is intended to differentiate between the two mutually exclusive alternatives.

The method of switching between network interfaces, for example from the first network interface to the second network interface may lead to an incomplete transmission of an imaging data at the first network interface. The incomplete transmission of an imaging data can generate an interrupt at the imaging station. In order to compensate for incomplete transmission of the imaging data, the method of communicating further comprises a step of retransmitting the imaging data upon receiving the interrupt as shown at step 235.

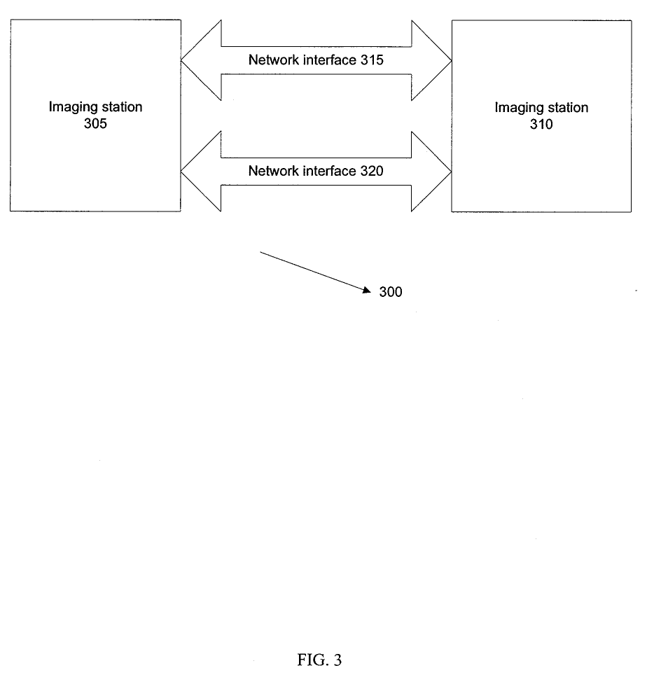

FIG. 3 provides a communication system 300 in another embodiment of the invention. The communication system 300 comprises multiple imaging stations shown at 305 and 310. The communication system 300 may comprise additional imaging stations (not shown). At least one imaging station 305 is configured to communicate with another imaging station 310 via a Digital Imaging and Communication in Medicine (DICOM) communication. The DICOM communications covered include, but are not limited to, Image Send/Transfer, DICOM Storage, DICOM Storage Commitment, DICOM Modality work list, and DICOM Print.

Each imaging station 305 and 310 comprises multiple network interfaces 315 and 320. Each network interface 315 and 320 can be one of a wired network interface and a wireless network interface. Each network interface 315 and 320 corresponds to a single network address. The attached network address may use any of several different formats, such as an AE title, IP address and a port number. A mapping of each network interface 315 and 320 with a corresponding network address is maintained as the configuration table at the imaging station 305. The imaging station 305 is configured to maintain and update any changes in the network address of any of the network interfaces. An unlimited number of network addresses may be stored in the configuration table. For example, between 50 and 100 network addresses may be stored in the configuration table.

The imaging station 305 is configured to automatically detect an available network interface, the available network interface being a network interface available for communication and select one of the available network interface. Upon selecting the network interface say a first network interface 315, the imaging station 305 configures itself to the corresponding network address of the first network interface 315 and communicates one or more imaging data via the first network interface 315. The imaging station 305 is further configured to switch between the network interfaces 315 and 320 as needed. The imaging station 305 is configured to automatically switch from the first network interface 315 to a second network interface 320 available for communication, and to automatically switch from communicating imaging data via the first network interface 315 to communicating imaging data via the second network interface 320 upon configuring itself to communicate via the second network interface 320. The detection of the available network interface and switching between the network interfaces 315 and 320 occurs as long as the imaging station 305 is running. An interrupt can be generated when a switch between the network interfaces 315 and 320 occurs followed by an unsuccessful transmission of the imaging data. The imaging station 305 is further configured to retransmit the imaging data upon receiving the interrupt.

In another embodiment, a computer program product stored in one or more computer readable media is provided. The computer program product comprises a method of communicating in a communication system 300 comprising an imaging station 305 with multiple network interfaces 315 and 320. The computer program product comprises a routine for selecting a first network interface 315 available for communication in the imaging station 305, a routine for configuring the imaging station 305 to communicate via the first network interface 315 and a routine for communicating one or more imaging data from the imaging station 305 via the first network interface 315. The computer program product further comprises a routine for selecting a second network interface 320 available for communication in the imaging station 305, and a routine for automatically reconfiguring the imaging station 305 to communicate via the second network interface 320. The routine for communicating may comprise a routine for retransmitting the imaging data upon receiving an interrupt. The computer program product may further comprise a routine for maintaining a configuration table, mapping each of the network interfaces with a corresponding network address.

In various embodiments, the invention relates to a method of communicating in a communication system by switching between network interfaces of a networked device such as an imaging station. More specifically, the invention provides a method for seamless switching of the network traffic between the network interfaces of a networked device. The method eliminates the need for an operator to switch between the network interfaces manually and reconfigure the networked device for a selected network interface. Hence the method improves workflow by providing seamless transition and automatic reconfiguration between network interfaces.

The seamless automatic switching and reconfiguration between network interfaces allows for continuous DICOM connectivity. A user may carry out operations like printing, sending images, sending storage commitment, etc, without waiting for switching between network interfaces.

The method also provides for automatically retransmitting the imaging data that is not completely transmitted when the network interface is switched. Thus there may be no loss of imaging data when the network interface is switched. The additional advantages of using the invention will be apparent to those of skill in the art.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of communicating in a communication system comprising an imaging station, the imaging station comprising multiple network interfaces, the method comprising:
selecting a first network interface available for communication in the imaging station, each network interface being identified with a network address;
configuring the imaging station to communicate via the first network interface; and
communicating at least one imaging data from the imaging station via the first network interface.

2. The method of claim 1, further comprising selecting a second network interface available for communication in the imaging station, and automatically reconfiguring the imaging station to communicate via the second network interface.

3. The method of claim 1, wherein the network interface is one of a wired network interface and a wireless network interface.

4. The method of claim 1, wherein the network address comprises at least one of an Internet Protocol (IP) address, an Application Entity Title (AETitle) and a port number.

5. The method of claim 1, further comprising maintaining a configuration table at the imaging station, wherein the configuration table comprises a mapping of each network interface with a corresponding network address.

6. The method of claim 1, wherein the method of communicating comprises retransmitting the imaging data upon receiving an interrupt, wherein the interrupt indicates an unsuccessful transmission.

7. A communication system comprising:
at least one imaging station comprising multiple network interfaces, each network interface being identified with a network address.

8. The communication system of claim 7, wherein the imaging station is configured to select a first network interface available for communication.

9. The communication system of claim 8, wherein the imaging station is further configured to communicate at least one imaging data via the first network interface.

10. The communication system of claim 9, wherein the imaging station is further configured to retransmit the imaging data upon receiving an interrupt, where the interrupt indicates an unsuccessful transmission.

11. The communication system of claim 8, wherein the imaging station is configured to automatically switch from the first network interface to a second network interface available for communication, and to automatically switch from communicating imaging data via the first network interface to communicating imaging data via the second network interface.

12. The communication system of claim 7, wherein the network interface is one of a wired network interface and a wireless network interface.

13. The communication system of claim 7, wherein the network address comprises at least one of an Internet Protocol (IP) address, an Application Entity Title (AETitle) and a port number.

14. The communication system of claim 7, wherein the imaging station is configured to maintain a configuration table, wherein the configuration table comprises a mapping of each network interface with a corresponding network address.

15. The communication system of claim 7, wherein at least one imaging station is configured to communicate with another imaging station via a Digital Imaging and Communication in Medicine (DICOM) communication

16. The communication system of claim 15, wherein each imaging station comprises one of an imaging server and an imaging modality.

17. The communication system of claim 16, wherein the imaging modality is one of a magnetic resonance imaging (MRI) system, computed tomography (CT) system, ultra sonography (US) system, nuclear medicine (NM) system, digital fluoroscopy (DF) system, digital radiography (DR) system and computer radiography (CR) system.

18. A computer program product, stored in one or more computer readable media for communicating in a communication system comprising an imaging station with multiple network interfaces, the computer program product comprising:
a routine for selecting a first network interface available for communication in the imaging station, each network interface being identified with a network address;
a routine for configuring the imaging station to communicate via the first network interface; and
a routine for communicating at least one imaging data from the imaging station via the first network interface.

19. The computer program product of claim 18, further comprising a routine for selecting a second network interface available for communication in the imaging station, and a routine for automatically reconfiguring the imaging station to communicate via the second network interface.

20. The computer program product of claim 18, further comprises a routine for maintaining a configuration table, wherein the configuration table comprises a mapping of each network interface with a corresponding network address.

21. The computer program product of claim 18, wherein the routine for communicating comprises a routine for retransmitting the imaging data upon receiving an interrupt, wherein the interrupt indicates an unsuccessful transmission.

22. The computer program product of claim 18, wherein the network interface is one of a wired network interface and a wireless network interface.

23. The computer program product of claim 18, wherein the network address comprises at least one of application entity title (AETitle), Internet protocol (IP) address and port number.
